(19) United States
(12) Patent Application Publication
(10) Pub. No.: US 2009/0060944 A1
(43) Pub. Date: Mar. 5, 2009

(54) Enhancer Of The Effect Of Adrenocorticoid,Comprising Gum Arabic

(76) Inventors: Michinori Kubo, Osaka (JP); Yumiko Kubo, Osaka (JP); Hideaki Matsuda, Osaka-fu (JP)

Correspondence Address: WENDEROTH, LIND & PONACK, L.L.P., 2033 K STREET N. W., SUITE 800, WASHINGTON, DC, 20006-1021, US

(21) Appl. No.: 11/667,690

(22) PCT Filed: Nov. 29, 2005
(86) PCT No.: PCT/JP05/22267

§ 371 Date: May 14, 2007

(30) Foreign Application Priority Data
Nov. 30, 2004 (JP) 2004-346673

(51) Int. Cl. A61K 9/34; A61P 13/12

(52) U.S. Cl. 424/195.18

(57) ABSTRACT

This invention provides an enhancer of the effect of adrenocorticoid on nephritis without enhancement of the undesirable side-effects of the said adrenocorticoid, comprising the water-soluble gum obtained from the stem and/or branch of Acacia species, e.g., Gum Arabic. The combination of the adrenocorticoid and the said water-soluble gum enhances the effect of the said adrenocorticoid on nephritis without enhancement of the undesirable side-effects of the hormone agent.

### TECHNICAL FIELD

The present invention relates to an enhancer of the effect of adrenocorticoids, especially an enhancer comprising a water-soluble gum from the stem and/or branch of Acacia species, which enhances the protective effect of the said adrenocorticoid on nephritis without enhancement of the undesirable side-effects of the adrenocorticoid.

### BACKGROUND ART

Adrenocortical hormones are biosynthesized in adrenal glands and secreted from the glands. The hormones are classified into two groups, namely mineralocorticoids such as aldosterones and deoxycorticosterones having electrolyte metabolic activity and glucocorticoids such as cortisone and hydrocortisone having liver glycogen activity or glucocorticoid activity. Since the latter group has a variety of pharmacological activities such as anti-allergic activity, anti-inflammatory activity and immuno-suppressive activity, many glucocorticoids have been synthesized.

As the results of extensive chemical investigation, several potent anti-inflammatory glucocorticoids, such as prednisolone, dexamethasone and betamethasone, have been developed. These synthetic agents are clinically used worldwide as anti-inflammatory agents. In this specification, adrenocorticoid means a natural and/or synthetic adrenocorticoid clinically used.

The long term use of adrenocorticoids and the large amount adrenocorticoids that is dosed give several undesirable problems, such as disorders of the gluco-metabolism, mineral-metabolism and protein-metabolism. As undesirable side-effects of adrenocortical hormones, increased infection, disorder of adrenal glands, diabetes mellitus, gastric ulcers, mental disorder, depression, osteoporosis, joint pains, moon faces, edema, hypertension, cataract, glaucoma, and retardation of wound healing are reported (Osaka Hospital Pharmacist Association Ed., “Iyakuhin Youran” 5th Ed. Yakuji-jihou-sha, Tokyo).

Nephritis is an inflammatory disorder of several tissues in kidneys such as glomerular, tubular, and interstitial tissue. These inflammatory disorders are the target of the said adrenocorticoid. On the other hand, nephritic syndrome, one of nephritis, is characterized by excretion of large amount of urinary protein. Nephritic syndrome is also the target of the said adrenocorticoid (Takahisa T., Kobayashi N., Ebihara A. Ed., “Zusetu; Rinshouyakuri to Kihonchiryouyaku”, Medical View Co. ltd.).

A known water-soluble gum is a gummy exudation obtained from the stems and/or branch of Acacia senegal (Leguminosae) or more undetermined Acacia species (Leguminosae). Acacia senegal originally comes from a dry region between Senegal and Ethiopia in Africa, and is now widely distributed in India. It grows as a small tree, to about 6 m in height, and has a gray smooth bark and a branch with zigzag shape. It is characterized by three thorns attached to each leaf, wherein the central thorn is curved, and having cream-colored flowers. Gum Arabic (GA) is a naturally collected resin, a colorless or pale yellow transparent ball-like lump. Sometimes it has a milky non-transparent appearance. Most commercially available GA used for industry is the ball-shaped gummy exudation collected from this species. Other gummy exudation collected from Acacia seyal is also commercially available.

The said gummy exudation obtained from the stems and/or branch of Acacia species has been used as paste since the pre-Christian era. It was called Gum Arabic since the said gummy exudation was produced in Africa and exported through Arabia to other foreign countries. GA is colorless or pale yellow, obtained as transparent ball-like lump/pieces, and sometimes has a milky non-transparent appearance. It has many cracks on the surface, and is easily broken; when broken, the new surface is glassy and often sheds luster. GA is odorless, tasteless and viscous.

The major component of GA is arabic acid (79 to 81%), which exists as Ca, Mg and/or K salts. Acid hydrolysis of GA yields L-arabinose, D-galactose, L-rhamnose, and D-glucuronic acid. In addition, trace of hydrolase and oxidase are present, together with a small amount of minerals and proteins.

GA has been used as a binder for fixing a pigment from the past, and still used as a binder for transparent and non-transparent water paints at present. In addition to water paints, GA is widely used as an emulsifying agent in confectionery, such as candies or soft drinks, and as an additive when manufacturing a sugar-coated tablet or syrup in pharmaceuticals. GA is a known material, and a binder comprising about 35% of GA is also used as a medium for giving transparency to a water paint and/or gloss to a picture painted with the water paint, as well as used as a binder when manufacturing the water paint. A 3% solution of GA is useful as a binder for a writing brush after that has been washed out.

From the era of ancient Egypt, GA has been used as a folk medicine for the treatment of periodontal disease and alveolar pyorrhea (treating bleeding from the gums, removing ulcers in the gums, or promoting growth of the teeth), lung disorder and liver disorder.

At present, GA is used as an emulsifier with flavor for keeping homogeneity of ingredients of juices and ice-creams, a food additive for maintaining the shape of candies and stabilizers of medicines for compressing tablets or preventing disproportionation of the ingredients in a liquid formulation.

Although it has been reported that Gum Arabic decreases blood cholesterol and triglyceride levels in human (Ross A H, Eastwood M A, Brydon W G, Anderson J R, Anderson D M, Am. J. Clin. Nutr., 37, 368-75, 1983), improves levels of intestinal flora (Phillips G O, Food Addit. Contam., 15, 251-264, 1998) and has a protective effect on acetaminophen-induced hepatotoxicity (Gamal el-din A M, Mostafa A M, Al-Shabanah O A, Al-Bekairi A M, Nagi M N, Pharmacological Research, 48, 631-635, 2003), the efficacy of Gum Arabic as an enhancer of the effect of adrenocorticoids in nephritis, and the efficacy of Gum Arabic as a reducer of the side-effects of adrenocorticoids have not been reported.

At the present time, there is no ideal therapy for nephritis. Several adrenocorticoids are used for the treatment of nephritis, but these hormones are accompanied by many side-effects. This invention offers a novel agent which reduces the said side-effects, and enhances the efficacy of adrenocorticoids without enhancement of the said side-effect.

### DISCLOSURE OF INVENTION

The inventors studied a gummy exudation from the stems and/or branch of Acacia species (Leguminosae), and found that the said gummy exudation enhances the efficacy of adrenocorticoids without enhancement of the side-effects and that the said gummy exudation reduces the side-effects of adrenocorticoids. Namely, this invention relates to an enhancer of the effect of adrenocorticoids by the combination of an adrenocorticoid and the said gummy exudation from the stem and/or branch of Acacia species.

In this specification, “adrenocorticoid” means the natural and/or synthetic adrenocorticoid clinically used in steroid therapy. Examples of adrenocorticoids include, but are not limited to, e.g., cortisone, hydrocortisone, prednisone, prednisolone, methylprednisolone, triamcinolone, dexamethasone, paramethasone, betamethasone, fluorometholone, flumethasone, fludrocortisone, beclomethasone, clobetasol, diflucortolone, clobetasone, diflorasone, alclometasone, triamcinolone acetonide, fluocinolone acetonide, fludroxycortide, fluocinonide, halcinonide, amcinonide, flunisolide, and budesonide.

As the water-soluble gum in this invention, a gummy exudation from the stems and/or branch of Acacia species (Leguminosae) such as Acacia senegal and Acacia seyal can be used in its intact form. From the viewpoint of easy availability and easy formulation, the dried powder of the said gum and/or the extract of the said gum are preferable.

The preferable extraction solvent is water. The aqueous extract is used as such, or after concentration, dilution and/or purification. The dried powder and/or the extract can be purified by means of column chromatography.

The said enhancer of this invention may be administered orally in the form of solution in water or hot water, or in the form of aqueous drink agent. The daily dose of the said enhancer depends, for example, on the patient's sex, age and body weight, and the dose of adrenocorticoid. The daily dose of the said enhancer is usually 1 to 100 g, preferably 10 to 50 g, more preferably 10 to 20 g.

If administered as a drink, if necessary, appropriate amount of a sweetener such as sugar, honey, glycerin or aspartame, spice such as garlic or ginger, perfume such as fruity flavor, antioxidant such as ethylenediamine disodium salt or sodium thiosulfate, amino acid such as leucine, methionine, lysine, or taurine, and vitamin such as vitamin A, vitamin B, vitamin C, vitamin D, vitamin E, or nicotinamide, may be added to the drink formulation.

Natural juice such as orange juice or grapefruit juice, and/or the extract of crude drug such as Ginseng and Young Deer Horn also can be added to the drink formulation for oral administration.

Nephritis as the target of the enhancer of this invention is nephritic syndrome such as lupus nephritis or lipoid nephritis, which is typically treated with an adrenocorticoid.

The adrenocorticoid includes, but not limited to, prednisolone, dexamethasone, betamethasone and methylprednisolone.

According to this invention, the amount of steroid that has to be administered to a host can be reduced, and the side-effects associated with the steroid therapy are successfully minimized.

Advantageous effects of this invention were confirmed by use of gentamicin-induced nephritis in rats.

### 1. Experiments

### 1) Materials

Gum Arabic was dissolved in water and filtered. The filtrate was spray-dried at about 120° C. to give the GA used in this experiment. GA was dissolved in tap water to prepare a 7.5% GA solution (7.5 g/100 mL, 7.5% GA). The 7.5% GA solution was used for oral administration in drinking water. Gentamicin sulfate (GM) was purchased from Sigma Chemical Co. and used as its saline solution. Prednisolone (PRD) was purchased from Sigma Chemical Co.

### 2) Animals

Male Wistar strain rats weighing 200-220 g (n=8 to 9 per group) were provided by SLC (Japan SLC, Hamamatsu). They were maintained in an air-conditioned room with lighting from 7 a.m. to 7 p.m. The room temperature (about 23° C.) and humidity (about 60%) were controlled automatically. They were kept for one week before treatment. Laboratory chows (Labo MR Stock, Nihon Nosan Kogyo K.K., Yokohama) and drinking water were freely available.

### 3) Induction of GM-Nephritis

Nephritis was induced in rats by daily intraperitoneal injections of GM (80 mg/kg, i.p.) at 10:00 a.m. for 8 consecutive days. Animals injected with saline (1 mL/kg, i.p.) served as normal control. GA (7.5% GA) via the drinking water was administered for 8 consecutive days.

Rats were randomly assigned to the following eight groups of 8 to 9 rats each:

Group 1 (Normal): received daily intraperitoneal injections of saline (1 mL/kg, i.p.) for 8 days with tap water as drinking water.

Group 2 (7.5% GA): received daily saline (1 mL/kg, i.p.) and 7.5% GA via drinking water for 8 days.

Group 3 (GM): received daily GM (80 mg/kg, i.p.) for 8 days with tap water as drinking water.

Group 4 (GM+7.5% GA): received daily GM (80 mg/kg, i.p.) and 7.5% GA via the drinking water for 8 days.

Group 5 (GM+PRD/water): received daily PRD/water (2 mg/kg, p.o.) at 1 hr before daily GM (80 mg/kg, i.p.) administration for 8 days with tap water as drinking water.

Group 6 (GM+PRD/water+7.5% GA): received daily PRD/water (2 mg/kg, p.o.) at 1 hr before daily GM (80 mg/kg, i.p.) administration and 7.5% GA via the drinking water for 8 days.

### 4) Determination

### Organ Wet Weight

On the last day, the organs (adrenal gland, thymus, spleen, kidney and liver) were isolated from the rats, and the ratio of each organ wet weight (mg)/body weight (100 g) was determined.

### Urine and Blood Collections

24 hr urine samples were obtained by keeping each animal in an individual metabolic cage for 24 hr at 10:00 a.m. on the 8th day. Rats were fasted for 1 day before urine collection. The collected urine was then centrifuged at 1,600×g for 10 min, and the supernatant was used for protein determination. Immediately after the urine collection, blood sample was taken from abdominal veins. The blood was centrifuged at 1,600×g for 10 min to give serum for the determination of blood urea nitrogen (BUN), serum creatinine and creatinine clearance.

### Determination of Urinary Protein, BUN, Serum Creatinine and Creatinine Clearance

The urinary protein excretion was determined by the method of Kingsbury et al (The Journal of Laboratory and Clinical Medicine, 11, 981-989, 1926) and expressed as mg/day. Briefly, a 3% solution of sulfosalicylic acid was added to a diluted urine sample, and placed at room temperature for 10 minutes. Then concentrations of urinary protein were determined from absorbance at 400 nm, and the urinary protein excretion (mg/day) was calculated from the volume of 24 hr urine. Commercial kits (Wako Pure Chemical Industries, Ltd.) were used to assay BUN, creatinine and creatinine clearance in serum with the following assay kits: Urea nitrogen B and Creatinine.

### Statistical Analysis

The experimental data were tested for statistical significant differences by means of Bonferroni/Dunn's method (Multiple Range Test).

### 2. Results

### 1) Effect of GA on GM-Induced Nephritis in Rats

Blood parameters [BUN and serum creatinine levels and creatinine clearance] (Table 1) and urine parameters (urine volume and urinary protein) (Table 2) of normal rats (Group 1), 7.5% GA-treated rats (Group 2), GM-treated rats (Group 3) and GM plus 7.5% GA-treated rats (Group 4) are shown.

TABLE 1
Effect of GA on BUN, creatinine and creatinine
clearance in GM-induced nephritis rats

| | | | Serum | Creatinine |
|---|---|---|---|---|
| | | BUN | creatinine | clearance |
| Groups | Treatments | (mg/dL) | (mg/dL) | (mL/min) |
| 1 | Normal | 17.3 ± 0.9 | 0.43 ± 0.08 | 1.72 ± 0.86 |
| 2 | 7.5% GA | 14.7 ± 1.8 | 0.46 ± 0.09 | 1.17 ± 0.60 |
| 3 | GM | 78.0 ± 11.9^{##} | 2.36 ± 0.41^{##} | 0.19 ± 0.10^{#} |
| 4 | GM + 7.5% | 56.6 ± 7.2* | 1.73 ± 0.32 | 0.28 ± 0.10 |
| | GA | | | |

Each value was determined at 8th day and represents the mean ± S.E. of 8 to 9 rats.
Significantly different from Group 1,
^{#}p < 0.05,
^{##}p < 0.01.
Significantly different from Group 3,
*p < 0.05.

TABLE 2
Effect of GA on urine volume and urinary
protein in GM-induced nephritis rats

| | | Urine volume | Urinary protein |
|---|---|---|---|
| Groups | Treatments | (mL/24 hr) | (mg/24 hr) |
| 1 | Normal | 28.9 ± 4.5 | 3.0 ± 0.4 |
| 2 | 7.5% GA | 22.3 ± 3.9 | 3.7 ± 0.6 |
| 3 | GM | 43.1 ± 4.3^{##} | 26.7 ± 3.3^{##} |
| 4 | GM + 7.5% GA | 13.6 ± 1.6** | 19.8 ± 1.0 |

Each value was determined at 8th day and represents the mean ± S.E. of 8 to 9 rats.
Significantly different from Group 1,
^{##}p < 0.01.
Significantly different from Group 3,
**p < 0.01.

As shown in three blood parameters (Table 1) and two urine parameters (Table 2), there was no difference between data of the normal rats (Group 1) and the 7.5% GA-treated rats (Group 2).

In Group 3, BUN and serum creatinine were significantly increased, and creatinine clearance was significantly decreased (Table 1). Urine volume and urinary protein were significantly increased (Table 2). These parameter shifts show GM-induced nephritis in the rats, and GA significantly inhibited the increase of BUN, and showed inhibitory tendencies on the increase of serum creatinine and decrease of creatinine clearance (Group 4 in Table 1). GA also significantly inhibited the increase of urine volume and showed an inhibitory tendency on the decrease of urinary protein (Group 4 in Table 2).

The organs (adrenal gland, thymus, spleen, kidney and liver) were isolated from the rats on the last day. The results of determination of the ratio of each organ wet weight (mg)/body weight (100 g) are given in Table 3.

TABLE 3
Effect of GA on several organ weights in GM-induced nephritis rats

| | | Adrenal gland | Thymus | Spleen | Kidney | Liver |
|---|---|---|---|---|---|---|
| | | (mg/100 g of | (mg/100 g of | (mg/100 g of | (g/100 g of | (g/100 g of |
| Groups | Treatments | body weight) | body weight) | body weight) | body weight) | body weight) |
| 1 | Normal | 21.5 ± 0.7 | 262.3 ± 15.8 | 204.1 ± 8.3 | 0.77 ± 0.02 | 2.96 ± 0.08 |
| 2 | 7.5% GA | 20.1 ± 0.9 | 246.7 ± 18.6 | 186.3 ± 3.8 | 0.76 ± 0.02 | 2.89 ± 0.07 |
| 3 | GM | 23.5 ± 1.4 | 223.5 ± 15.2 | 209.4 ± 10.8 | 0.89 ± 0.03^{##} | 2.95 ± 0.07 |
| 4 | GM + 7.5% GA | 23.3 ± 0.8 | 237.1 ± 11.2 | 222.8 ± 8.3 | 0.89 ± 0.02 | 3.02 ± 0.05 |

Each value was determined at 8^{th} day and represents the mean ± S.E. of 8 to 9 rats.
Significantly different from Group 1,
^{##}p < 0.01.

As shown in Table 3, kidney weight ratio was slightly increased in GM-treated (Group 3) and GM plus 7.5% GA-treated rats (Group 4) as compared to normal (Group 1) and 7.5% GA-treated rats (Group 2). GA had no inhibitory effect on increments of kidney weight ratio. The other organ weights did not show any differences.

### 2) Effect of Combination of GA and PRD on GM-Induced Nephritis in Rats

PRD (2 mg/kg, p.o.) was orally administered in a form of suspension (0.2 mL/100 g weight of rat) in tap water (PRD/water) 1 hr before GM (80 mg/kg, i.p.) administration to rats of group 5 (GM+PRD/water) and group 6 (GM+PRD/water+7.5% GA). Group 5 received tap water as drinking water. Group 6 received 7.5% GA as drinking water.

Three serum parameters at the last day are given in Table 4.

TABLE 4
Effect of combination of Gum Arabic (GA) and prednisolone
(PRD) on BUN, serum creatinine and creatinine clearance
in GM-induced nephritis rats

| | | | Serum | Creatinine |
|---|---|---|---|---|
| | | BUN | creatinine | clearance |
| Groups | Treatments | (mg/dL) | (mg/dL) | (mL/min) |
| 1 | Normal | 17.3 ± 0.9 | 0.43 ± 0.08 | 1.72 ± 0.86 |
| 3 | GM | 78.0 ± 11.9 | 2.36 ± 0.41 | 0.19 ± 0.10 |
| 5 | GM + PRD/ | 44.8 ± 7.6** | 1.42 ± 0.32* | 0.46 ± 0.17 |
| | water | | | |
| 6 | GM + PRD/ | 25.1 ± 42**,^{§} | 0.70 ± 0.14**,^{§} | 1.14 ± 0.57 |
| | water + | | | |
| | 7.5% GA | | | |

Each value was determined at 8^{th} day and represents the mean ± S.E. of 8 to 9 rats.
Significantly different from Group 3,
*p < 0.05,
**p < 0.01.
Significantly different from Group 5,
^{§}p < 0.05.

GM markedly increased BUN level as shown in Group 3 of Table 4. All PRD-treated (2 mg/kg, p.o.) groups (Groups 5 and 6) showed a significant protective effect on increments of BUN level in the GM-induced nephritis rats. Combination of PRD and GA examined in PRD/water plus 7.5% GA-treated group (Group 6) in Table 4 significantly enhanced this protective effect of PRD on BUN compared with PRD/water-treated group (Group 5). A significant enhancement of the protective effect was also found in serum creatinine. With respect to creatinine clearance, a tendency to enhance the recovery caused by PRD was found, and thus, the combination of PRD and GA enhanced the protective effect of PRD on GM-induced nephritis in a synergistic manner.

Results of determination of urine volume and urinary protein at the last day are shown in Table 5.

TABLE 5
Effect of combination of Gum Arabic (GA) and
prednisolone (PRD) on urine volume and urinary
protein in GM-induced nephritis rats

| | | Urine volume | Urinary protein |
|---|---|---|---|
| Groups | Treatments | (mL/24 hr) | (mg/24 hr) |
| 1 | Normal | 28.9 ± 4.5 | 3.0 ± 0.4 |
| 3 | GM | 43.1 ± 4.3 | 26.7 ± 3.3 |
| 5 | GM + PRD/water | 42.3 ± 4.0 | 18.0 ± 3.1* |
| 6 | GM + PRD/water + | 13.3 ± 1.7**,^{§§} | 16.5 ± 3.1** |
| | 7.5% GA | | |

Each value was determined at 8^{th} day and represents the mean ± S.E. of 8 to 9 rats.
Significantly different from Group 3,
*p < 0.05,
**p < 0.01.
Significantly different from Group 5,
^{§§}p < 0.01.

GM increased both urine volume and urinary protein, and PRD significantly inhibited the increment of urinary protein. PRD-treated group (Group 5) did not show any effects on the increment of urine volume.

In PRD/water plus 7.5% GA-treated group (Group 6), increments of urine volume and urinary protein were significantly inhibited, and thus, the combination of PRD and GA enhanced the protective effect of PRD on GM-induced nephritis in a synergistic manner.

Results of determination of organ weight ratio are shown in Table 6.

TABLE 6
Effect of combination of Gum Arabic (GA) and prednisolone (PRD)
on several organ weights in GM-induced nephritis rats

| | | Adrenal | | | | |
|---|---|---|---|---|---|---|
| | | gland | Thymus | Spleen | Kidney | Liver |
| Groups | Treatments | (mg/100 g of body weight) | | | (g/100 g of body weight) | |
| 1 | Normal | 21.5 ± 0.7 | 262.3 ± 15.8 | 204.1 ± 8.3 | 0.77 ± 0.02 | 2.96 ± 0.08 |
| 3 | GM | 23.5 ± 1.4 | 223.5 ± 15.2 | 209.4 ± 10.8 | 0.89 ± 0.03 | 2.95 ± 0.07 |
| 5 | GM + PRD/water | 22.0 ± 0.8 | 166.2 ± 10.5** | 217.3 ± 8.7 | 0.89 ± 0.01 | 3.11 ± 0.06 |
| 6 | GM + PRD/water + | 20.8 ± 0.3* | 175.1 ± 14.0* | 189.8 ± 9.1^{§} | 0.91 ± 0.02 | 3.05 ± 0.07 |
| | 7.5% GA | | | | | |

Each value was determined at 8^{th} day and represents the mean ± S.E. of 8 to 9 rats.
Significantly different from Group 3,
*p < 0.05,
**p < 0.01.
Significantly different from Group 5,
^{§}p < 0.05.

In GM-treated rats (Group 3), the weight of thymus showed a tendency to decrease compared to the normal group (Group 1). In PRD-treated rats, the weight of thymus was further decreased (Group 5). PRD causes many side-effects of which reduction of thymus is known to be one. The combination of PRD and GA showed a small recovery of the thymus weight but it was not significant (Group 6). This means that a side-effect of PRD on thymus reduction was not enhanced by the combination of PRD and GA.

### EXAMPLE

### Preparation of Drink Agent

The water-soluble gum (10 g) was dissolved in hot water (40° C., 30 mL) with stirring. After cooling the solution to 25° C., vitamin B1 (10 mg), vitamin B6 (10 mg), caffeine (50 mg), sugar (5 g), honey (5 g), citric acid (400 mg), sodium citrate (50 mg), and sodium benzoate (35 mg) were added with stirring. The pH of the resulting solution was adjusted to 6.0 by addition of lactic acid and 0.1 N sodium hydroxide. Then the total volume of the resulting solution was adjusted to 50 mL by addition of water.

The water-soluble gum used in this Example was prepared as follow: a Gum Arabic obtained from Acacia senegal in Sudan was powdered and dissolved in water. Then the resulting solution was filtered and the filtrate was spray-dried to give the said water-soluble gum.

### INDUSTRIAL APPLICATION

This invention offers a useful therapeutic method for the treatment of nephritis by increasing the utility of adrenocorticoid therapy, because the present invention enhances the effect of adrenocorticoids without enhancement of the undesirable side-effect of adrenocorticoids.

1. Enhancer of the effect of adrenocorticoid comprising a water-soluble gum obtained from the stem and/or branch of Acacia species.

2. Enhancer of the effect of adrenocorticoid of claim 1, wherein the said adrenocorticoid is used for the treatment of nephritis.

3. Enhancer of the effect of adrenocorticoid of claim 1, wherein the said water-soluble gum is Gum Arabic.

4. Enhancer of the effect of adrenocorticoid of claim 1, wherein the said Acacia species is Acacia senegal.

5. A product containing an adrenocorticoid and a water-soluble gum obtainable from the stem and/or branch of Acacia species, as a combined preparation for simultaneous, separate or sequential use in steroid therapy.

6. A product according to claim 5, wherein the therapy is of nephritis.

7. A product according to claim 5, wherein the gum is Gum Arabic.

8. A product according to claim 5, wherein the Acacia species is Acacia Senegal.

9. (canceled)

10. (canceled)

11. (canceled)

12. (canceled)

13. A method for the treatment of a condition responsive to steroid therapy, which comprises administering to a patient in need thereof, in addition to a steroid, a water-soluble gum obtainable from the stem and/or branch of Acacia species.

14. A method according to claim 13, wherein the therapy is of nephritis.

15. A method according to claim 13, wherein the gum is Gum Arabic.

16. A method according to claim 13, wherein the Acacia species is Acacia Senegal.
